(19) United States
(12) Patent Application Publication
(10) Pub. No.: US 2026/0306737 A1
(43) Pub. Date: Oct. 8, 2026

(54) Medicament delivery system and method therefor

(71) Applicant: SHL Medical AG, Zug (CH)

(72) Inventors: Thomas Daniel James, Baltimore, MD (US); Florence Olivia Stevenson, Baltimore, MD (US); Stephen Allison Porter, Baltimore, MD (US); Mariano Mumpower, Baltimore, MD (US)

(21) Appl. No.: 19/480,426

(22) PCT Filed: Apr. 24, 2024
(86) PCT No.: PCT/EP2024/061175

§ 371 Date: Oct. 31, 2025

Related U.S. Application Data
(60) Provisional application No. 63/464,767, filed on May 8, 2023.

(30) Foreign Application Priority Data
Jun. 13, 2023 (EP) 23178885.2

(51) Int. Cl. A61M 5/142; A61M 5/148; A61M 5/168

(52) U.S. Cl. CPC A61M 5/14212; A61M 5/1486; A61M 5/16881; A61M 2205/3337; A61M 2205/3368

(57) ABSTRACT

The present disclosure provides a medicament delivery system including a cassette comprising a medicament bag, a connector, and a pump unit connectable to the cassette via the connector. The pump unit is configured to introduce air in the cassette around the medicament bag. The pump unit includes a pump, an air inlet, an air valve, and a pressure sensor configured to measure pressure in the cassette. The pump unit defines an air inlet path between the air inlet and the connector, said air inlet path comprising the pump. The pump unit further defines an air return path between the connector and the air valve, when said air valve is open.

### CROSS-REFERENCE TO RELATED APPLICATIONS

The present application is a U.S. National Phase Application pursuant to 35 U.S.C. § 371 of International Application No. PCT/EP2024/061175 filed Apr. 24, 2024, which claims priority to (i) U.S. Provisional Application No. 63/464,767 filed May 8, 2023, and (ii) EP Patent Application No. 23178885.2 filed Jun. 13, 2023. The entire disclosure contents of these applications are herewith incorporated by reference into the present application.

### TECHNICAL FIELD

The present disclosure relates to the field of medicament delivery systems.

### BACKGROUND

Some drugs are required to be stored in a refrigerator for reasons of storage life and for the medicament not to lose its properties. However, in general and also for automatic and semi-automatic delivery systems, the drug should reach ambient temperature to render the delivery more comfortable since the injection of a cold substance can be painful. Moreover, the temperature also affects the viscosity for some medicaments.

In an autoinjector, the patient should typically let the drug warm at room temperature for about 30 minutes. However, depending on the volume of the medicament and the packaging, this step might take longer than expected or desired.

When delivering large volumes contained within a bag in a cassette, this may be a problem as the air pocket between the bag and atmosphere may insulate the drug and warm it up slower than when it is contained in a glass vial.

One conventional way to measure the temperature to know when the drug is ready for injection is to put some type of electronics in or close to the medicament or some type of temperature sensitive label on the medicament container. However, these solutions will add cost and complexity and also negatively affect the sustainability.

It is thus an object of the present disclosure to remedy the deficiencies of the prior art.

### SUMMARY

According to an aspect, a (partially) reusable medicament delivery device/system is provided.

According to an aspect, a warm-up time of the drug may be reduced.

According to an aspect, medicament delivery may be more comfortable for a patient.

Moreover, methods and systems according to the present disclosure are provided to speed up the warming up of the drug as well as to measure the temperature of the drug and indicate when it is ready for injection.

The invention is specified by the independent claims. Preferred embodiments are defined in the dependent claims. In the following description, although numerous features may be designated as optional, it is nevertheless acknowledged that all features comprised in the independent claims are not to be read as optional.

According to a first aspect, the invention relates to a medicament delivery system comprising:
a cassette comprising a medicament bag;
a connector;
a pump unit connectable to the cassette via the connector, configured to introduce air in the cassette around the medicament bag, the pump unit comprising a pump, an air inlet, an air valve and a pressure sensor defining:
   an air inlet path between the air inlet and the connector, said air inlet path comprising the pump;
   an air return path between the connector and the air valve, when said air valve is open;
wherein the pressure sensor is configured to measure pressure in the cassette.

Thus, the present disclosure encompasses a medicament delivery system having a pneumatic circuit with separate inlet and return paths, in which inlet and outlet of the pump unit meet at the cassette connection, i.e., the connector. Through this separate inflow and outflow path, a continuous air flow can be provided to warm up the interior of the cassette and thus the medicament contained in the medicament bag. Alternatively, by closing the air valve, the cassette may be pressurised, and the medicament expelled from the cassette.

In a nom-limited embodiment, the cassette comprises at least one baffle plate in the interior thereof, configured to distribute and/or guide air introduced into the interior of the cassette from the air inlet path to the air return path.

In a nom-limited embodiment, the baffle plate comprises at least a ridge in the interior of the cassette.

In a nom-limited embodiment, the pump unit comprises a processing unit configured to control the pump and the air valve according to the measured pressure.

In a nom-limited embodiment, the pressure sensor is located between the pump and the connector.

In a nom-limited embodiment, the pressure sensor is located between the air valve and the connector.

In a nom-limited embodiment, the processing unit is configured to determine a change of temperature of the interior of the cassette based on the measured pressure.

According to a second aspect, the invention relates to a method for warming up a medicament bag of a medicament delivery system according to the first aspect of the invention, comprising:
by means of the pump, providing a continuous air flow in the interior of the cassette from the air inlet path to the air return path, the air valve being open, the temperature of the air flow being higher than the temperature of the air inside the cassette (1);
by means of the pressure sensor, measuring the pressure in the cassette.

In a non-limited embodiment, the method further comprises:
when said pressure is constant, stopping the continuous air flow and closing the air valve.

In a non-limited embodiment, the method further comprises:
determining a change of temperature of the interior of the cassette based on the measured pressure.

In a non-limited embodiment, the temperature change is determined based on the ideal gas law, preferably according to the Gay-Lussac Law.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction of a longitudinal extension of the device which, in the figures, extends along the x-axis.

Similarly, the terms “lateral”, “laterally”, “transverse”, “transversal” and “transversally” refer to a direction generally perpendicular to the longitudinal direction (x-axis) which is marked as the y-axis in the figures.

The terms “vertical” and “vertically” refer to a direction perpendicular to a plane spanned by the longitudinal and lateral direction (x-y-plane) which is marked as the z-axis in the figures.

Further, the terms “circumference”, “circumferential”, or “circumferentially” refer to a circumference or a circumferential direction relative to an axis. Similarly, “radial” or “radially” refer to a direction extending radially relative to the axis, and “rotation”, “rotational” and “rotationally” refer to rotation relative to the axis.

The term “separable” in accordance with the present invention means that the pump unit can be physically separated from the cassette. A separable pump unit can also be carried on or affixed to the cassette.

The term “independent” in accordance with certain embodiments of the present invention means that the pump unit is functionally independent from the cassette and from the medicament bag(s). The pump unit according to the present invention can thus co-operate with a plurality of different cassettes, as desired by the user, wherein the cassette is adapted to communicate with a pump unit. Thus, the user is provided with several options as regards the use of the pump unit. If desired, however, the independence of the pump unit can be deactivated or disabled or at least restricted so that a given pump unit is operable with only one or a limited number of cassettes.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1A shows an embodiment of a medicament delivery system according to the present disclosure in a pre-assembly state.

FIG. 1B shows the medicament delivery system according to FIG. 1A in an assembly state.

FIG. 1C shows the medicament delivery system according to FIG. 1A or 1B in an assembled state.

FIG. 2A shows a sectional view of a cassette according to an embodiment of the present disclosure.

FIG. 2B shows another sectional view of the cassette according to FIG. 2A.

FIGS. 3A to 3C show perspective views of an interior of a pump unit according to an embodiment of the present disclosure.

FIG. 4 shows a sectional view schematically illustrating an air flow path according to an embodiment of the present disclosure.

FIG. 5 shows a schematic diagram of an air flow path according to an embodiment of the present disclosure.

FIG. 6 shows exemplary graphs of a warm-up phase of a cassette according to an embodiment of the present disclosure.

FIG. 7 shows exemplary graphs of a pressure change during a warm-up phase of a cassette according to an embodiment of the present disclosure.

### DETAILED DESCRIPTION

All elements and embodiments described below with reference to the drawings may be combined unless indicated otherwise.

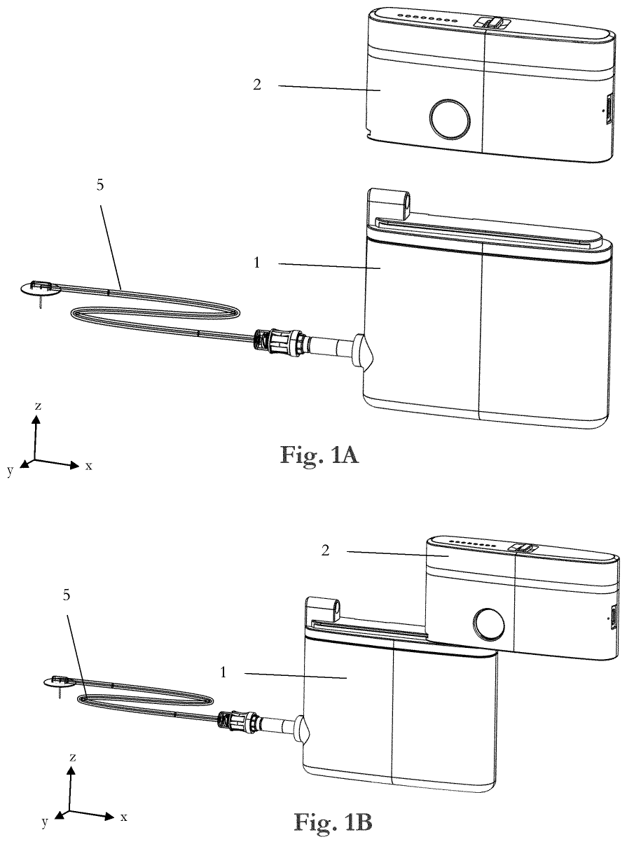

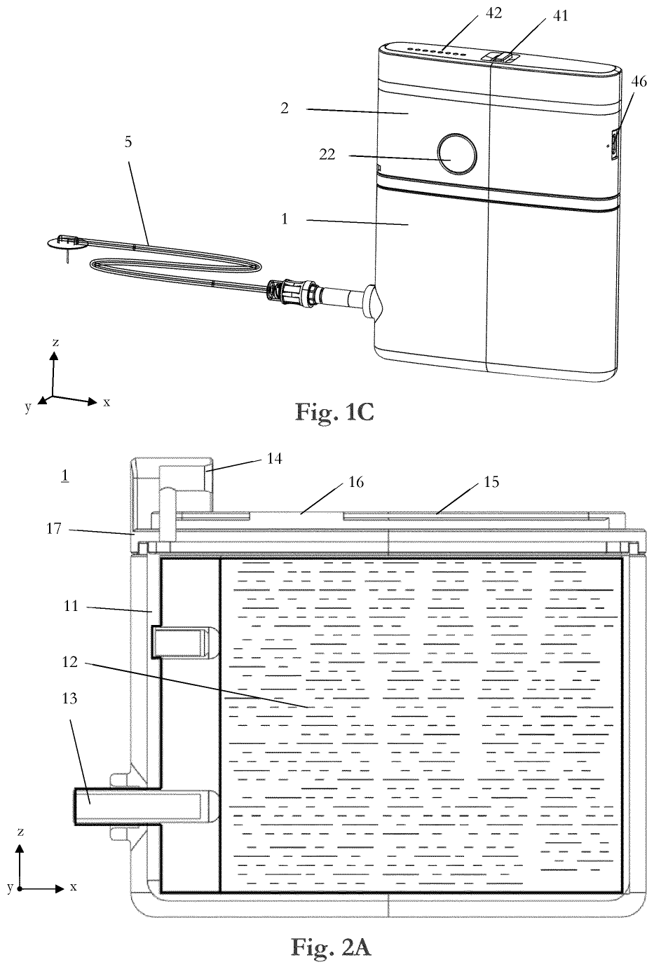

FIG. 1A shows an overview of a medicament delivery system according to an embodiment of the present disclosure. The system comprises a pump unit 2 and a cassette 1. The cassette 2 may be connectable to an infusion set 5. For example, a standard Luer connection may be employed. However, other types of infusion sets and connections can be used with the present disclosure. The cassette 1 and the pump unit 2 may be separately provided and connectable to each other.

FIG. 1B shows the pump unit 2 and the cassette 1 (with an infusion set 5 attached) during assembly or connection of both parts. The pump unit 2 is slid onto the cassette 1 or vice versa. In FIG. 1B, a sliding engagement between the pump unit 2 and the cassette 1 is shown but this is merely an example and not to be understood as limiting the disclosure.

FIG. 1C shows the medicament delivery system in an assembled state. The pump unit 2 has been attached to the cassette 1 and both parts are locked with respect to each other. The device is thus ready for medicament delivery. After medicament delivery, the pump unit 2 can be detached or separated from the cassette by pushing the release button 22 on the pump unit 2.

The pump unit 2 may thus be separable from the cassette 1. In particular, the pump unit 2 may be functionally independent from the cassette 1 and from the medicament bag(s) 12. The pump unit 2 according to the present invention can thus co-operate with a plurality of different cassettes 1, as desired by the user, wherein the cassette 1 is adapted to communicate with a pump unit 2. The pump unit 2 may also be configured to be connectable to only one (type of) cassette 1.

In a top view, both the pump unit 2 and the cassette 1 may have an oval shape. In particular, the pump unit 2 and the cassette 1 may have a similar or the same shape in an x-y-plane. In the assembled state, the circumferences of the pump unit 2 and the cassette 1 may be flush with respect to each other. The vertical extension of the pump unit 2 and cassette 1 may differ depending on the electronics and parts provided in the pump unit 2 and the kind and volume of drug (i.e., the medicament bag 12) provided in the cassette 1.

Various parts of the system will now be described in more detail.

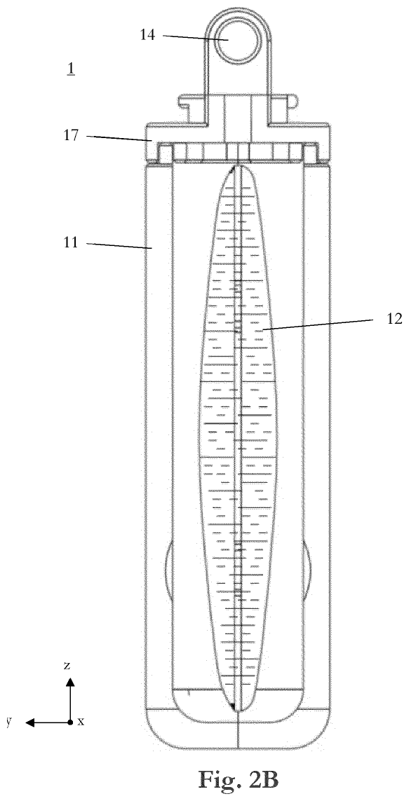

FIG. 2A shows a sectional view of a cassette 1 according to an embodiment along a longitudinal direction (x-z-plane). The cassette 1 comprises an outer shell 11 which may be formed as a single hard shell or as a plurality of shell parts assembled together. The outer shell 11 or the shell parts may be closed by a cassette lid 17. Inside the outer shell 11, a medicament bag 12 containing a medicament (drug) is provided. The medicament may be liquid, in particular at the time of dispensing. The medicament bag 12 may be a flexible (collapsible) medicament bag 12. A volume of the medicament bag may be, e.g., between 10 ml and 250 ml, preferably between 50 ml and 100 ml. The medicament bag 12 has an outlet port 13 which may also be used for filling the medicament bag 12 during production. The outlet port 13 may reach through a respective opening in the outer shell 11. The outlet port 13 may be provided with a valve, such as a pinch valve, to allow medicament to flow out only if the valve is opened, e.g., by attaching a infusion set 5. The cassette 1 may further comprise an entry passage 14 allowing a fluid, in particular air, to enter the inside of the cassette 1.

The cassette 1 and the medicament bag 12 may be single use units or may be refillable.

In order to attach and secure the pump unit 2 to the cassette 1, a cassette locking mechanism 15 may be provided. The locking mechanism 15 may be provided on the cassette lid 17. In the figures, a guide rail 15 of a slide-on mechanism is shown. However, the pump unit 2 and the cassette 1 may also be connectable by a snap-fit, clamping, screwing or any other suitable means of connection. The locking mechanism 15 may also comprise locking cut-out 16 to be described later.

The cassette 1 is, apart from the entry passage 14, provided in an air-tight manner. That is, the outer shell 11 or outer shell parts and the cassette lid 17 are connected such that the interior of the cassette 1 is sealed. Consequently, the opening through which the outlet port 13 passes is also sealed in order not to allow air to pass.

FIG. 2B is a further sectional view of the cassette according to FIG. 2A along a lateral direction (y-z-plane). As can be seen from the figure, between the outer shell 11, the cassette lid 17, i.e., the interior of the cassette 1, and the medicament bag 12, a space is provided. For medicament delivery, the interior of the cassette 1 is pressurised by introducing a fluid, in particular air, through the entry passage 14 to the space. Thereby, the pressure acts on the collapsible medicament bag 12 and forces the medicament through the outlet port 13 for medicament delivery. This process will be described in more detail below.

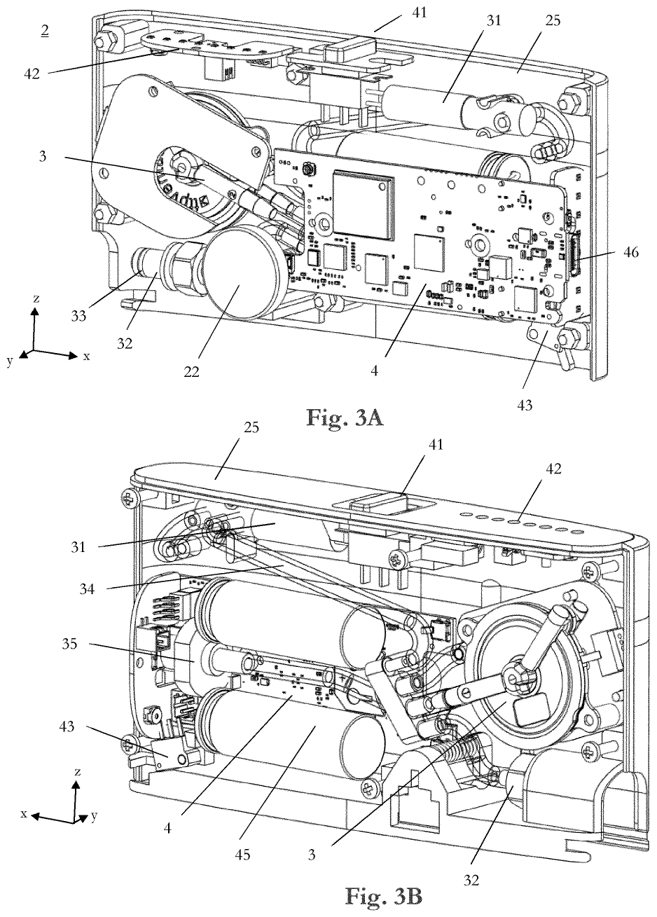

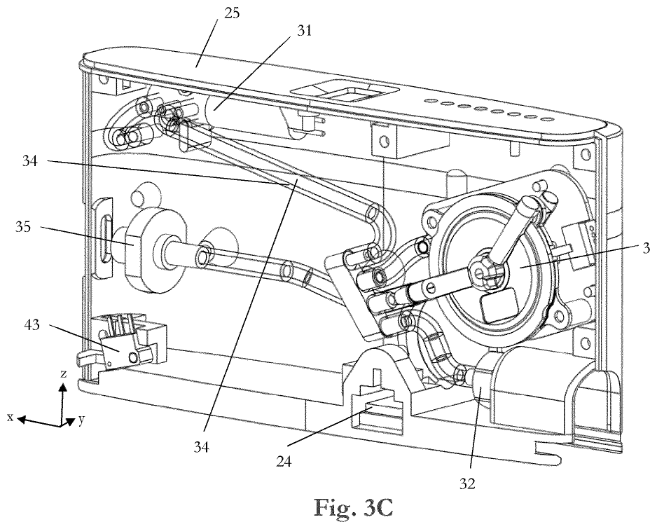

FIG. 3A is a perspective view of a pump unit 2 according to an embodiment. In the embodiments shown in the figures, the pump unit 2 comprises two shell parts 25 but it may also consist of one single shell part 25 or more than two shell parts 25. In FIGS. 3A to 3C, the shell part 25 facing the viewer has been omitted to allow inspection of the interior.

The medicament delivery system comprises a connector 32 between the pump unit 2 and the cassette 1. The connector 32 serves as an air inlet/outlet for pressurising/de-pressuring the cassette 1. The connector 32 may be provided with a sealing member 33. The sealing member 33 may be an O-ring fitted into a recess in the connector 32 as shown in FIG. 3A. The sealing member 33 may thus be radially compressed when a cassette 1 is attached to the pump unit 2 to seal the connection between the connector 32 and the entry passage 14 as will be further described below.

The pump unit 2 comprises a pump 3, particularly an air pump 3. The pump unit 2 further comprises an air valve 31. The air valve 31 and the connector 32 may be connected by a tubing 34.

The pump unit 2 further comprises electronics such as a processing unit 4 for controlling the medicament delivery device. The processing unit 4 may also be referred to as a control unit. The processing unit 4 may comprise at least one processor, a memory, at least one power source 45 and a communication interface 46. The communication interface 46 may be used for charging the power source 45 and for connecting the pump unit 2 to a computer or a server. The pump unit 2 may, in addition or alternative to the power source 45, also comprise a disposable battery. In addition, a wireless communication module may be provided.

The pump unit 2 may also comprise an identification unit (not shown) configured to identify the type of cassette 1 attached to the pump unit 2, i.e., parameters or prescription particulars of the drug such as type, volume, temperature for delivery, speed of delivery, time of delivery, etc. Thus, the cassette 1 may comprise an identification tag allowing the pump unit 2 to identify the cassette 1. This may be achieved, e.g., via RFID, NFC or the like. It may be preferred that the identification tag at the cassette 1 is a passive component.

The processing unit 4 may control operation of the pump 3 and the air valve 31. Moreover, the processing unit 4 may be connected to a power switch 41 for starting a medicament delivery or switching the device on or off, an indicator 42 for indicating various states of the device and a connection switch 43 for detection whether a cassette 1 is attached to the pump unit 2. The indicator 42 may comprise at least one illumination means such as an LED and/or a sound generator or speaker. The indicator 42 may provide visual and/or audible feedback of the state of the device which may comprise at least one of an active state of the processing unit 4, an attachment state of the cassette 1, an operational state, a battery state, a medicament temperature, a pressure state, an error state, a type of drug, whether the device is ready for injection, or whether the injection has been successful. The pump unit 2 may further comprise at least one pressure sensor and/or at least one temperature sensor (not shown). The pressure sensor may be provided at a suitable spot of the air flow path, e.g., at the connector 32, the pump 3, the air valve 31 or the tubing 34. The temperature may be measured, e.g., at an air inlet 35, the connector 32, the pump 3 or the air valve 31 or in between at the tubing 34. Specifically, the temperature of the incoming air may be measured and/or the temperature of air flowing out of the cassette 1 in order to obtain data of a medicament temperature inside the cassette 1. Also, a medicament temperature may be directly measured.

FIG. 3B is a perspective view of the pump unit 2 from the other side of the device, i.e., rotated 180° around the vertical z-axis. Again, the shell part 25 facing the viewer has been omitted in the figure.

The pump unit 2 further comprises an air inlet which may comprise the air inlet 35. The air inlet 35 is also connected to the pump 3 via a tubing.

In FIG. 3C showing the same perspective view as FIG. 3B, most of the features apart from the air flow path have been omitted. For medicament delivery, ambient air is sucked in by the pump 3 via the air inlet 35 and provided to an attached cassette 1 via the connector 32. During that process, the air valve 31 is closed in order to pressurise the cassette 1. When the pressure inside the cassette 1 is to be released, the air valve 31 is opened and the air flows to the outside via the tubing 34 and the air valve 31. The air inlet 35 and air outlet (i.e., the air valve 31) may be provided inside the pump unit 2 which may not be hermetically sealed. Alternatively, openings may be provided in the shell parts 25 allowing air to pass or connecting the air inlet 35 and the air outlet (i.e., the air valve 31) to the outside.

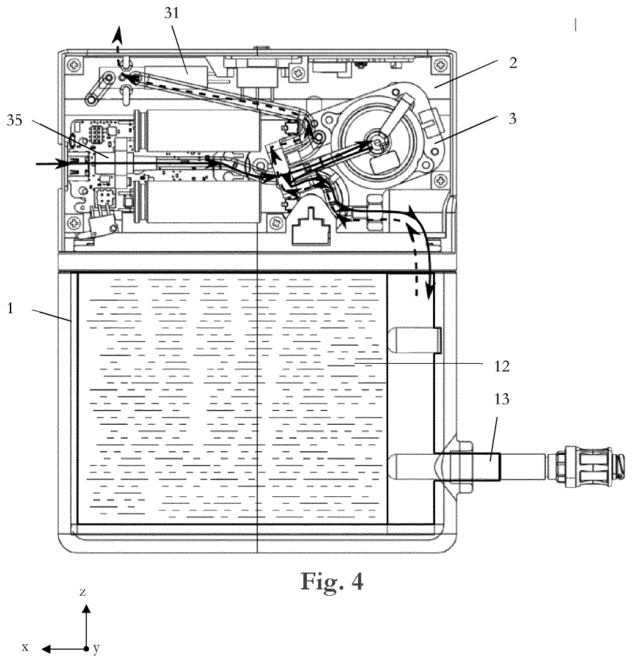

FIG. 4 shows the air flow inside the device according to an embodiment. Therein, the solid line indicates the inflow air, and the dashed line indicates the outflow air. For medicament delivery, the air valve 31 is closed, and ambient air is sucked in by the pump 3 through the air inlet 35 and the tubing 34 and then provided to the cassette 1 via the tubing 34, the connector 32 and the entry passage 14. By pumping air into the cassette 1, the interior of the cassette 1 is pressurised, wherein the pressure acts on the medicament bag 12 to force the medicament into the infusion set. In other words, the pressure in the interior of the cassette 1 exerts a force on the medicament bag 12, in particular on the outer surface of the medicament bag 12. Thereby, the medicament is pushed out of the medicament bag 12 through the outlet port 13.

In case air is to be released from the cassette 1, the air valve 31 is opened, and the interior of the cassette 1 may be de-pressurised through the entry passage 14, the connector 32, the tubing 34 and the air valve 31.

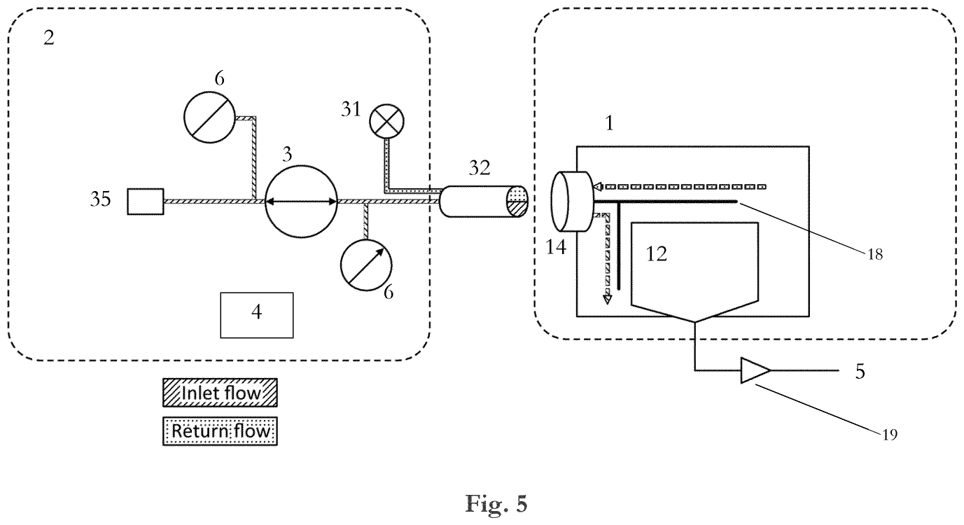

FIG. 5 is a further schematic diagram of a medicament delivery system according to the invention, and air flow paths (inlet flow path and return flow path) during a method for warming up the medicament in the cassette 1 of the medicament delivery system, according to an embodiment. As described above, the pump unit 2 comprises a pump 3 and an air inlet 35 connected to the pump 3. The pump unit 2 comprises an air valve 31 as well, also referred to as a blowoff valve. The pump unit 2 may comprise a controller or processing unit 4 configured to control the pump 3 and the air valve 31. The pump unit 2 comprises at least one pressure sensor 6. The pressure sensor 6 may measure the pressure of a fluid in the interior of the cassette 1. The fluid may be air. The at least one pressure sensor 6 may be connected to and controlled by the processing unit 4 (controller). In addition, a temperature sensor (not shown) may be provided. The pressure sensor 6 may be located after the pump 3 in the direction of air flow, that is to say between the connector 32 and the pump 3. The pressure sensor 6 may alternatively be located between the connector 32 and the air valve 31.

The pump unit 2 may be connected to the cassette 1 via the connector 32 and the entry passage 14 as described above. The cassette 1 may comprise flow routing baffle plates 18 for guiding or directing an air flow inside the cassette 1. An infusion set 5 may be connected to the cassette 1, e.g., via a check valve with crack pressure 19.

The connection between the connector 32 and the entry passage 14 may be designed such that a separate air inlet and return path is provided. E.g., the connector 32 may be split by a separator. A path from the pump 3 (inflow path) and a path to the air valve 31 (outflow path) may be separated by the separator.

As explained above, in the cassette 1, at least one flow routing baffle plate 18 may be provided to direct the air flow coming from the pump 3 through the connector 32 and the entry passage 14 such that an effective air distribution inside the cassette 1 is achieved. This is schematically depicted in FIG. 5 (but omitted in the previous figures). The baffle plates 18 may be formed by ridges or similar structures in the interior of the cassette 1. The baffle plates 18 may extend transversally, i.e., in the y-direction from one inner wall of the cassette 1 to the other.

As described above, during medicament delivery, the cassette 1 is pressurised by the pump 3 pumping air into the interior of the cassette and de-pressurised by opening the (blowoff) air valve 31.

In an embodiment of a method of (accelerated) warming of the drug according to the invention, prior to the medicament delivery, forced convection can be used with constant flow.

Thus, the present disclosure encompasses a medicament delivery system having a pneumatic circuit with separate inlet and return paths, in which inlet and outlet of the pump unit 2 (drive assembly) meet at the cassette 1 connection, i.e., the connector 32 of the pump unit 2 and the entry passage 14 of the cassette 1. This is shown in the diagram of FIG. 5, with the connection between the pump unit 2 and the cassette 1 represented by a male radial sealing interface.

The air valve 31 is normally closed, preferably latching, during the medicament delivery, but open for the convection-based drug warming method according to the invention. With a separate inlet and return path, warm air is pumped into the cassette 1 and allowed to exit via the return path, through the air valve 31. The cassette 1 may contain baffling or ducting features 18 as outlined above which force the inlet air over a large portion of the drug container (medicament bag 12) to maximize heat transfer.

Through this separate inflow and outflow path, a continuous air flow can be provided, if desired, to warm up the interior of the cassette 1 and thus the medicament contained in the medicament bag 12. Alternatively, by closing the air valve 31, the cassette may be pressurised.

A temperature of the interior of the cassette 1 may be determined as follows.

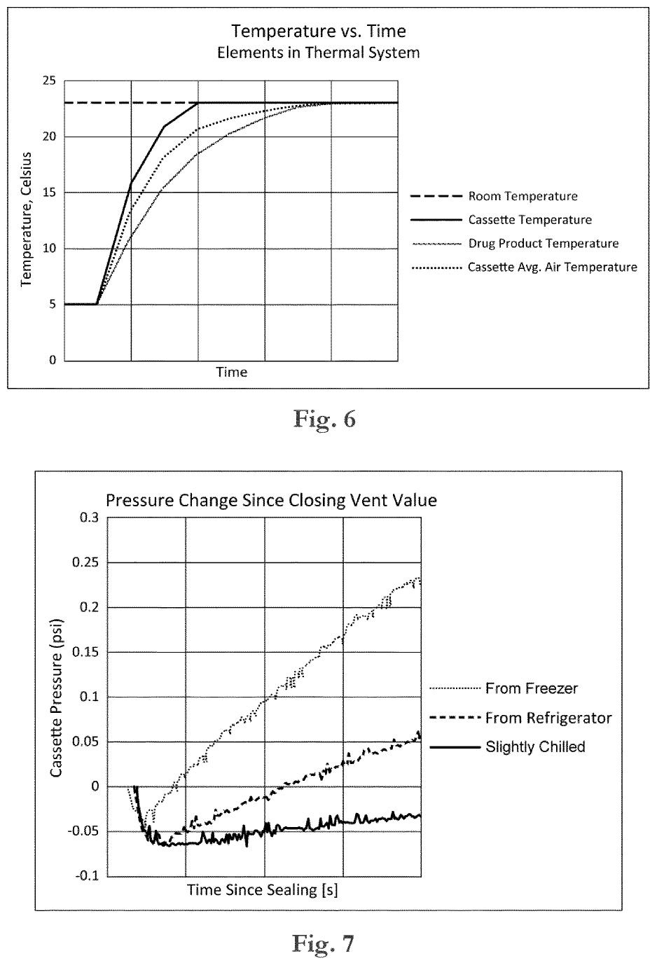

When removed from refrigeration, initially, both the drug product contained in the medicament container 12 and the air contained in the cassette 1 will be at the refrigeration temperature. As the enclosure of the cassette 1 warms to room temperature, the air in cassette 1 will serve as an insulator between the shell 11 of the cassette 1 (cassette enclosure) and the medicament container 12 surface. As air has a low thermal mass, it may reach steady state between the two surfaces relatively quickly. As the cassette enclosure 11 is in contact with the ambient air, it may warm more quickly than the drug product in the container 12. However, over time, the enclosed drug product will too equilibrate to room temperature, whether or not the method of “accelerated” heat transfer and warming described herein is implemented. This behaviour is illustrated in FIG. 6, with the cassette warming quickest, followed by the internal air, and last, the drug product.

In particular, in FIG. 6, the dashed line indicates the room temperature, the leftmost thicker line the cassette 1 temperature, the middle line the cassette 1 average air temperature and the rightmost line the drug temperature in the medicament container 12 over time. As can be seen from the graph, the cassette 1 reaches room temperature the quickest, followed by the internal temperature of the cassette 1 and the medicament container 12, i.e., the drug.

As described above, the medicament delivery system may be provided with a pressure sensor 6 at a suitable position. Thus, since the system is fitted with a pressure sensor 6 connected to the cassette's 1 air volume (although preferably residing in the pump unit 2 as indicated above), it is possible to detect the presence of a temperature change in the net temperature of the air in the cassette 1 by measuring the cassette 1 air pressure, using the Ideal Gas Law, or more specifically, Gay-Lussac's Law. The determination or monitoring of the pressure or pressure change in the interior of the cassette 1 may be performed by the at least one pressure sensor 6 or the processing unit 4 which may be connected to the at least one pressure sensor 6. As the fluid path check valve may prevent fluid outflow from the drug container 12, the volume inside the cassette 1 is considered constant at sufficiently low pressures. Once the drug product temperature (i.e., the temperature of the medicament bag 12) has reached steady state, a pressure reading of the cassette 1 will be constant. In the inverse, prior to the drug reaching steady state, the drug temperature, and thus the bulk air temperature are both changing, equilibrating to ambient, resulting in a changing pressure signal from the constant-volume system. FIG. 7 shows an empirically-measured example of a warm-up phase occurring in a functionally equivalent pneumatic system. Therein, the cassette 1 pressure over time is indicated.

In FIG. 7, the upper graph indicates a pressure change of the interior of a cassette 1 taken from a freezer. As can be seen, the pressure change exhibits the steepest slope due to the large temperature difference between the ambient air and the drug container 12. Consequently, the middle graph showing the pressure change over time of a cassette 1 taken from a refrigerator is less steep and the gradient of the lower graph indicating the pressure change of a cassette 1 slightly chilled is even smoother.

Using this method, it is possible to detect thermal equilibrium in the drug cassette 1 without the need for a direct local temperature measurement. Once adequate equilibrium is detected, the controller 4 may then allow drug delivery to commence. The air valve 31 is then closed.

The figures of the present disclosure refer to a slide-on mechanism between the pump unit 2 and the cassette 1. Such a slide-on mechanism may facilitate sealing of both units with respect to each other due to the engagement of the connector 32 and the entry passage 14. However, other mechanisms such as snap-fit, screw-on, or the like may be provided as long as an air-tight connection allowing the pump 3 to pressurise the cassette 1 is ensured.

Also, air inlet and air outlet may be separately provided in the cassette 1.

The method described above may be performed with a medicament delivery system as previously described.

The present disclosure may also encompass a pump unit 2 and/or a cassette 1 for use with the described system and/or method. In particular, the present disclosure encompasses a pump unit 2 independent from a cassette 1.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1(GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C—C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3,5+2,7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-12. (canceled)

13. A medicament delivery system comprising:
a cassette comprising a medicament bag;
a connector; and
a pump unit connectable to the cassette via the connector, wherein the pump unit is configured to introduce air in the cassette around the medicament bag, wherein the pump unit comprises a pump, an air inlet, an air valve, and a pressure sensor configured to measure pressure in the cassette, the pump unit defining:
an air inlet path between the air inlet and the connector, said air inlet path comprising the pump; and
an air return path between the connector and the air valve, when said air valve is open.

14. The medicament delivery system according to claim 13, wherein the cassette comprises at least one baffle plate in the interior thereof, configured to distribute and/or guide air introduced into the interior of the cassette from the air inlet path to the air return path.

15. The medicament delivery system according to claim 14, wherein the baffle plate comprises at least a ridge in the interior of the cassette.

16. The medicament delivery system according to claim 13, wherein the pump unit comprises a processing unit configured to control the pump and the air valve according to the measured pressure.

17. The medicament delivery system according to claim 13, wherein the pressure sensor is located between the pump and the connector.

18. The medicament delivery system according to claim 13, wherein the pressure sensor is located between the air valve and the connector.

19. The medicament delivery system according to claim 13, wherein the processing unit is configured to determine a change of temperature of the interior of the cassette based on the measured pressure.

20. The medicament delivery system according to claim 19, wherein the temperature change is determined based on the ideal gas law, preferably according to the Gay-Lussac Law.

21. A method for warming up the medicament bag of the medicament delivery system according to claim 13, comprising:
by means of the pump, providing a continuous air flow in the interior of the cassette from the air inlet path to the air return path, the air valve being open, the temperature of the air flow being higher than the temperature of the air inside the cassette; and
by means of the pressure sensor, measuring the pressure in the cassette.

22. The method according to claim 21, further comprising:
when said pressure is constant, stopping the continuous air flow and closing the air valve.

23. The method according to claim 21, further comprising:
determining a change of temperature of the interior of the cassette based on the measured pressure.

24. The method of claim 23, wherein the temperature change is determined based on the ideal gas law, preferably according to the Gay-Lussac Law.
